(19) United States
(12) Patent Application Publication
(10) Pub. No.: US 2004/0156793 A1
(43) Pub. Date: Aug. 12, 2004

(54) Skin care composition and method of skin treatment

(75) Inventors: Mary Catherine Golden, Chesterfield, MO (US); Bernard Benjamin, Marina Del Rey, CA (US)

Correspondence Address: Pitney, Hardin, Kipp & Szuch, 685 Third Avenue, New York, NY, 10017, US

(73) Assignee: Golden Chiropractic Group Ltd.

(21) Appl. No.: 10/364,543

(22) Filed: Feb. 11, 2003

(51) Int. Cl. A61K 9/00

(52) U.S. Cl. 424/47

(57) ABSTRACT

A skin care composition including petrolatum and at least one selected from the group consisting of a C₅-C₇ alkane and a paraffin can be applied from a remote location to ameliorate skin conditions. The skin care composition may include zinc oxide when used to treat diaper rash.

This invention relates to skin care compositions and methods of treating a skin condition. Remarkably, though the skin care composition does not include propellant it can be delivered in a spray form.

### BACKGROUND OF THE INVENTION

Bodily functions such as urination and defecation can result in irritation of the skin. The absorbency of products such as diapers and disposable undergarments used to capture the products of bodily functions has increased over time. As a result, consumers of such products may have the products of bodily functions in contact with their skin surfaces for extended periods. The skin surface can become irritated and red and a rash may form leading to constant discomfort. The discomfort can be exacerbated upon touching the skin surface to apply medicaments. Further, touching the skin surface might lead to the spread of bacteria or even illness.

Accordingly, there is a need for a skin care composition that can be delivered to skin surfaces remotely without touching the surface. Unfortunately, most spray compositions require propellants in order to be delivered from a remote location. Propellants in addition to being environmentally undesirable, may have health implications for those in the immediate vicinity of their discharge.

### SUMMARY OF THE INVENTION

Accordingly it is an object of the invention to provide a skin care composition which is environmentally desirable and helps prevent spread of infection.

Another object of the invention is to provide a method of treating a skin condition by delivering a skin treatment composition to skin surfaces remotely without touching the skin surface wherein the skin treatment composition is not hazardous to the environment.

These and other objects of the invention are achieved by providing a skin care composition including petrolatum and at least one selected from the group consisting of a C₅-C₇ alkane and a paraffin wherein the composition has a viscosity such that it can be delivered in a spray form from a vessel fitted with a manually operated spray device and wherein the skin care composition is free of propellant. In this manner the skin care composition can be applied from a remote location without actually touching the skin surface.

### DETAILED DESCRIPTION OF THE PREFERRED EMBODIMENTS

The present invention provides for a skin care composition including petrolatum and at least one selected from the group consisting of a C₅-C₇ alkane and a paraffin wherein the composition has a viscosity such that it can be delivered from a vessel fitted with a manually operated spray device and wherein the skin care composition is free of propellant. Preferably the skin care composition is pourable at room temperature., i.e. 75 degrees fahrenheit, and the at least one selected from the group consisting of a C₅-C₇ alkane and a paraffin has a boiling point less than 380 degrees fahrenheit.

The petrolatum is present in an amount of 5% to 95% by weight, preferably 20% to 80% by weight, most preferably 30% to 60% by weight of the skin care composition. A suitable petrolatum is White Propet #2L available from Witco.

The skin care composition also includes at least one selected from the group consisting of a C₅-C₇ alkane and a paraffin, present in an amount of less than 50% by weight, preferably 9% to 25% by weight of the skin care composition. The C₅-C₇ alkane can be linear or branched or cyclic. A preferred C₅-C₇ alkane is hexane. A suitable hexane is available from Ashland or Vopak or Chemcentral.

Paraffin is present in the skin care composition in the amount of 5% to 50% by weight, preferably 20% to 50% by weight of the skin care composition. The term paraffin as used herein is a hydrocarbon obtained from petroleum having more than seven carbon atoms and has a boiling point less than 400 degrees fahrenheit. Preferably, the paraffin is an isoparaffin with less than 0.0003 weight percent aromatics or no aromatic character. An isoparaffin is a paraffin with one or more points of branching along the hydrocarbon chain. Suitable isoparaffins include but are not limited to Isopar C, Isopar E and Isopar G available from Exxon Mobil. A preferred paraffin is Isopar G.

The combination of petrolatum and at least one selected from the group consisting of a C₅-C₇ alkane and paraffin results in an emulsion having a viscosity such that it can be poured at room temperature and can be delivered in a spray form from a vessel fitted with a manually operated spray device such as a pump type actuator. Most preferably once sprayed, at least a portion of one or more components selected from the group consisting of a C₅-C₇ alkane and paraffin evaporates, and the viscosity of the skin composition sprayed on a target surface increases such that the skin composition does not run off the target surface.

The skin care composition may also contain zinc oxide which is useful in treatment of diaper rash. The zinc oxide is present in the amount of up to 40% by weight, preferably 0.5% to 25% by weight, most preferably 5% to 15% by weight of the skin care composition. A suitable zinc oxide is USP-1 grade available from Zinc Corporation of America.

The skin care composition may also include anti-wrinkle and anti-aging actives, anti-microbial actives, anti-viral actives, anti-fungal actives, anti-acne actives, anti-eczema actives, anti-inflammatory actives, vitamin actives, protein actives, and mixtures thereof. In general the amount of active varies from about 0.0001% to about 50% by weight of the skin care composition.

The anti-wrinkle and the anti-aging actives include without limitation hydroxy acids including C₂-C₃₀ alpha-hydroxy acids such as glycolic acid, lactic acid, 2-hydroxy butanoic acid, malic acid, citric acid, tartaric acid, alpha-hydroxyethanoic acid, hydroxycaprylic acid and the like; beta hydroxy acids including salicylic acid and polyhydroxy acids including gluconolactone (G4); and mixtures of these acids. Further anti-wrinkle agents include retinoic acid, gamma-linolenic acid; fruit acids, sugar cane extract and glycomer in cross-linked alpha nutrium, and mixtures thereof.

Antimicrobials that may be used in accordance with the present invention include all antibiotics, antimicrobial agents and antimicrobial peptides. Antibiotics that may be used include dermatologically acceptable salts of tetracylin and tetracyclin derivatives, gentamycin, kanamycin, streptomycin, neomycin, capreomycin, lineomycin, paromomycin, tobramycin, erythromycin, triclosan, octopirox, parachlorometa xylenol nystatin, tolnafiate, miconazole hydrochloride, chlorhexidine gluconate, chlorhexidin hydrochloride, methanamine hippurate, methanamine mandelate, minocycline hydrochloride, clindamycin, cloecin, b-lactam derivatives such as aminopenicillin, chlorhexidin gluconate, and tricolosan and mixtures thereof.

Anti-acne actives that may be used in accordance with the present invention include without limitation keratolytic agents including lactic acid, pyruvic acid, salicylic acids, urea and N-acetylcysteine; retinoids, and retinoid analogs such as tretinoin, cis and trans retinoic acid, retinol and retinol palmitate, isotretinoin-13-cis-retinoic acid; antibiotics and antimicrobial agents such as tetracycline, erythromycin, minocycline, clindamycin, trimethoprim-sulphamethazole and anti-microbial peptides (nicin, for example); steroids, such as hydrocortisone, gamma-linolenic acid and mixtures thereof. Further anti-acne actives that may be used include without limitation benzoyl peroxide; alpha and beta hydroxy acids; sulfacteamide and sulfur and mixtures thereof. Preferably used herein are salicylic acid, benzoyl peroxide and retinoids.

Anti-psoriasis actives preferred for use in the present invention include without limitation salicylic acid; mometasone furoate; steroids including corticosteroids such as cortisone and oluxclobetasol propionate; 5-fluorouracil; epinephrine; anthralin, vitamin D3 analogs, such as calcipotriene, methotrexate; masprocol; trimethaxate gluconate; retinoids, cyclosporin; paclitaxel, 5-amino levulinic acid; bergasol; tin-ethyl etio purpurin; benzoporphyrin derivatives; antibodies, such as ABX-IL8 antibody, CD11a monoclonal antibody and ICM3 monoclonal antibody; enzyme inhibitors, including tryptase inhibitor and phospholipase A-2 inhibitors; angiogenesis blocking agents; T-cell blocking agents and mixtures thereof.

Anti-eczema actives useful herein include urea; evening primrose oil; plant extracts; hydrocortisone; an immunomodulator; tar combined with fatty acids obtained from banana; and mixtures thereof.

Topical anesthetic actives that may be used in accordance with the present invention include tetracaine, lidocaine, editocaine, bupivacaine, pramoxine; and mixtures thereof.

Anti-inflammatory actives useful in accordance with the present invention include steroidal actives such as hydrocortisone as well as non-steroidal actives including propioinic derivatives; acetic acid derivatives; biphenylcarboxylic acid derivatives, fenamic acid derivatives; and oxicams. Example of anti-inflammatory actives include without limitation acetaminophen, oxaprozin, pranoprofen, benoxaprofen, bucloxic acid, elocon; and mixtures thereof.

Vitamin actives which may be used in accordance with the present invention include vitamin A and derivatives, including retonic acid, retinyl aldehyde, retin A, retinyl palmitate, adapalene, beta-carotene; vitamin B (panthenol, provitamin B5, panthenic acid, vitamin B complex factor); vitamin C (ascorbic acid and salts thereof) and derivatives such as ascorbyl palmitate; vitamin D including calcipotriene (a vitamin D3 analog) vitamin E including its individual constituents alpha-, beta-, gamma-, delta-toco-pherol and cotrienols and mixtures thereof and vitamin E derivatives including vitamine E palmitate, vitamin E linolate and vitamin E acetate; vitamin K and derivatives; vitamin Q (ubiquinone) and mixtures thereof. Proteins and peptides which may be used in accordance with the present invention include enzymes such as proteases (e.g. bromelain, papain, collagenase, elastase), lipases (e.g. phospholipase C), esterases, glucosidases, exfoliating enzymes; antibodies and antibody derived actives, such monoclonal antibodies, polyclonal antibodies, single chain antibodies and the like; reductases; oxidases; peptide hormones; natural structural skin proteins, such as elastin, collagen, reticulin and the like; growth factors such as platelet derived growth factor (PDGF) and epidermis derived growth factor (EGF); anti-oxidants such as superoxide dismutase, catalase and glutathione; free-radical scavenging proteins; DNA-repair enzymes, for example T4 endonuclease 5 and P53; antimicrobial peptides, such as magainin and cecropin; a milk protein; a silk protein or peptide; and any active fragments, derivatives of these proteins and peptides; and mixtures thereof an anti-viral agent (such as acyclovir); an anti-hemorrhoid compound, an anti-wart agent (such as podophyllotoxin) and a plant extract and mixtures thereof.

Anti-oxidants that may be incorporated herein include natural anti-oxidants prepared from plant extracts including without limitation extracts that may be obtained from aloe vera; cryocytol; avocado; chamomile; echinacea; ginko biloba; ginseng; green tea; heather; jojoba; lavender; lemon grass; licorice; mallow; oats; peppermint; St. John's wort; willow; wintergreen; wheat wild yam extract; marine extracts; and mixtures thereof. Further anti-oxidants that may be used include vitamins, including vitamin C, vitamin E and vitamin E mimetics; alpha-lipoic acid, coenzyme Q; glutathione; superoxide dismutase; and mixtures thereof.

In addition the skin care composition may include fragrances which are not irritating to the skin. The skin composition may further contain less than 50% by weight, preferably less than 20% by weight, mineral oil. Most preferably the skin care composition is free of surfactants or rheology modifiers.

The following Examples are illustrative only and do not limit the invention.

### EXAMPLE 1

89.3 pounds of Isopar G to and 89.3 pounds of hexane were added to a stainless steel container of a Cowles type high speed mixer. 197 pounds of petrolatum was melted and added to the container under agitation. After approximately 20 minutes the mixture appeared to be homogenous and 0.9 pounds of Perfume 168201 available from Belmay, Inc. was added to the mixture.

A ⅝ inch diameter marble available from JaBo, Parkersburg, W. Va. was inserted into a 3-piece tinplate unlined aerosol can size 202×406 (6 oz) available from Crown Cork & Seal Inc. and U.S. Can Company. 83.15 grams of the above mixture was introduced into the can. A valve was inserted into the can and fixed into place by pressing it firmly on the valve cup. A metal ring added to the bottom of a valve crimper head facilitate pressing the valve cup onto the can. The pump actuator used was a P4 Santos actuator 0.18 ml dosage, 0.018 MBU insert and dip tube cut for 6 oz can available from Precision Valve (Canada) Ltd. located in Ontario, Canada.

### EXAMPLE 2

138.6 pounds of Isopar G and 86.6 pounds of hexane were added to a stainless steel container of a Cowles type high speed mixer. 129.0 pounds petrolatum was melted and added to the container under agitation. After approximately 20 minutes the mixture appeared to be homogeneous and 54.4 pounds of zinc oxide USP-1 was added to the mixture. The mixture was agitated for another 20 minutes at which point 0.98 Perfume 68285 was added to the mixture.

A ⅝ inch diameter marble available from JaBo, Parkersburg, W. Va. was inserted into a 3-piece tinplate unlined aerosol can size 202×406 (6 oz) available from Crown Cork & Seal Inc. and U.S. Can Company. 90.0 15 grams of the above mixture was introduced into the can. A valve was inserted into the can and fixed into place by pressing it firmly on the valve cup. A metal ring added to the bottom of a valve crimper head facilitate pressing the valve cup onto the can. The pump actuator used was a P4 Santos actuator 0.18 ml dosage, 0.018 MBU insert and dip tube cut for 6 oz can available from Precision Valve (Canada) Ltd. located in Ontario, Canada.

The above description is illustrative and not limiting. Further modifications will be apparent to one of ordinary skill in the art in light of the disclosure and appended claims.

We claim:

1. A skin care composition comprising:
petrolatum and at least one selected from the group consisting of a C₅-C₇ alkane and a paraffin wherein the composition has a viscosity such that it can be delivered in spray form from a vessel fitted with a manually operated spray device and wherein the skin care composition is free of propellant.

2. A skin care composition according to claim 1 comprising petrolatum and a C₅-C₇ alkane.

3. A skin care composition according to claim 1 comprising petrolatum and a paraffin.

4. A skin care composition according to claim 1 comprising petrolatum, a C₅-C₇ alkane and a paraffin.

5. A skin care composition according to claim 1 wherein the C₅-C₇ alkane is hexane

6. A skin care composition according to claim 2 wherein the C₅-C₇ alkane is hexane.

7. A skin care composition according to claim 4 wherein the C₅-C₇ alkane is hexane.

8. A skin care composition according to claim 1 further comprising zinc oxide.

9. A skin care composition according to claim 2 further comprising zinc oxide.

10. A skin care composition according to claim 3 further comprising zinc oxide

11. A skin care composition according to claim 4 further comprising zinc oxide.

12. A skin care composition according to claim 5 further comprising zinc oxide.

13. A skin care composition according to claim 6 further comprising zinc oxide.

14. A skin care composition according to claim 7 further comprising zinc oxide.

15. A skin composition according to claim 1 comprising at least one selected from the group consisting of anti-wrinkle and anti-aging actives, anti-microbial actives, anti-viral actives, anti-fungal actives, anti-acne actives, anti-eczema actives, anti-inflammatory actives, vitamin actives and protein actives.

16. A method of treating a skin condition comprising:
spraying a skin composition onto the surface of skin, the skin care composition including petrolatum and at least one selected from the group consisting of a C₅-C₇ alkane and a paraffin wherein the composition has a viscosity such that it can be delivered from a vessel fitted with a manually operated spray device and wherein the skin care composition is free of propellant.

17. A method of treating a skin condition according to claim 16 wherein the skin care composition comprises petrolatum and a C₅-C₇ alkane.

18. A method of treating a skin condition according to claim 16 wherein the skin care composition comprises petrolatum and paraffin.

19. A method of treating a skin condition according to claim 16 wherein the skin care composition comprises petrolatum, a C₅-C₇ alkane and paraffin.

20. A method of treating a skin condition according to claim 17 wherein the C₅-C₇ alkane is hexane.

21. A method of treating a skin condition according to claim 20 wherein the C₅-C₇ alkane is hexane

22. A method of treating a skin condition according to claim 16 wherein the skin care composition further includes zinc oxide.

23. A method of treating a skin condition according to claim 17 wherein the skin care composition further includes zinc oxide.

24. A method of treating a skin condition according to claim 18 wherein the skin care composition further includes zinc oxide.

25. A method of treating a skin condition according to claim 19 wherein the skin care composition further includes zinc oxide.

26. A method of treating a skin condition according to claim 20 wherein the skin care composition further includes zinc oxide.

27. A method of treating a skin condition according to claim 21 wherein the skin care composition further includes zinc oxide.

28. A method according to claim 16 wherein the skin composition further includes at least one selected from the group consisting of anti-wrinkle and anti-aging actives, anti-microbial actives, anti-viral actives, anti-fungal actives, anti-acne actives, anti-eczema actives, anti-inflammatory actives, vitamin actives and protein actives.

29. A method according to claim 16 wherein the skin surface is the surface of buttocks.

30. A method according to claim 16 wherein the skin surface is the surface of an anus.

31. A method of treating diaper rash comprising:
spraying a skin composition onto the surface of skin exhibiting diaper rash, the skin care composition including petrolatum, hexane, zinc oxide and paraffin wherein the composition has a viscosity such that it can be delivered in spray form from a vessel fitted with a manually operated spray device and wherein the skin care composition is free of propellant.

32. A method of ameliorating diaper rash comprising:
spraying a skin composition onto the surface of skin susceptible to diaper rash, the skin care composition including petrolatum, hexane, zinc oxide and paraffin wherein the composition has a viscosity such that it can be delivered in spray form from a vessel fitted with a manually operated spray device and wherein the skin care composition is free of propellant.
